(19) United States
(12) Patent Application Publication
(10) Pub. No.: US 2007/0265655 A1
(43) Pub. Date: Nov. 15, 2007

(54) EMBOLIC PROTECTION FILTER WITH ENHANCED STABILITY WITHIN A VESSEL

(75) Inventors: Richard J. Renati, Los Gatos, CA (US); Timothy Stivland, Plymouth, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/382,423

(22) Filed: May 9, 2006

(51) Int. Cl. A61M 29/00

(52) U.S. Cl. 606/200

(57) ABSTRACT

Embolic protection filtering devices and methods for making and using the same. An example filtering device includes a filter wire, a filter including a support member coupled to the filter wire, and a filter membrane coupled to the support member. A traction member may be coupled to the support member. The traction member may help to stabilize the longitudinal position of the filtering device within a body lumen.

### FIELD OF THE INVENTION

The present invention pertains to embolic protection filtering devices. More particularly, the present invention pertains to embolic protection filtering devices with position-stabilizing features and characteristics.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences because the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

A wide variety of filtering devices have been developed for medical use, for example, intravascular use. Of the known filtering devices, each has certain advantages and disadvantages. There is an ongoing need to provide alternative filtering devices as well as alternative methods for manufacturing filtering devices.

### BRIEF SUMMARY

This disclosure pertains to design, material, and manufacturing method alternatives for filtering devices. An example filtering device includes a filter wire, a filter including a support member coupled to the filter wire, and a filter membrane coupled to the support member. A traction member may be coupled to the filter, for example, at or near the support member. The traction member may help to stabilize the longitudinal position of the filtering device within a body lumen.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is partial cross-sectional side view of an example filtering device disposed in a blood vessel;

FIG. 2 is a side view of the example filtering device shown in FIG. 1;

FIG. 3 is a side view of another example filtering device;

FIG. 4 is a side view of another example filtering device;

FIG. 5 is a side view of another example filtering device;

FIG. 6 is a side view of the filtering device depicted in FIG. 5 with an alternatively shaped traction member;

FIG. 7 is an alternative side view of either of the filtering devices depicted in FIGS. 5-6;

FIG. 8 is a side view of another example filtering device; and

FIG. 9 is an alternative side view of the filtering device depicted in FIG. 8.

### DETAILED DESCRIPTION

The following description should be read with reference to the drawings wherein like reference numerals indicate like elements throughout the several views. The detailed description and drawings illustrate example embodiments of the claimed invention.

When a clinician performs an intravascular intervention such as angioplasty, atherectomy, and the like, embolic debris may dislodge from the blood vessel that can travel in the bloodstream to a position where it may impair blood flow, possibly leading to tissue damage. A number of other situations and/or interventions may also result in the mobilization of embolic debris. Accordingly, embolic protection filtering devices have been developed that can be disposed in the blood vessel downstream of the treatment site and expanded to capture debris.

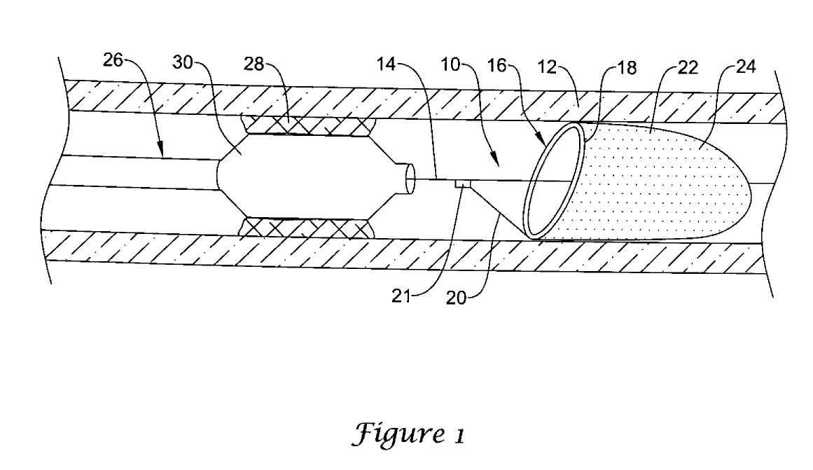

FIG. 1 is a partial cross-sectional view of an example embolic protection filtering device 10 disposed within a blood vessel 12. Filtering device 10 can be delivered to a suitable target region, for example within blood vessel 12, using an appropriate delivery device (not shown) and removed after use with a suitable retrieval device (not shown). Device 10 may include an elongate shaft or wire 14 having an embolic protection filter 16 coupled thereto. Filter 16 includes a filter loop or support member 18 and a filter membrane or fabric 22 coupled to support member 18. Filter membrane 22 can be drilled (for example, formed by known laser techniques) or otherwise manufactured to include a plurality of openings 24. These holes or openings 24 can be sized to allow blood flow therethrough but restrict flow of debris or emboli floating in the body lumen or cavity.

In general, filter 16 may be adapted to operate between a first generally collapsed configuration and a second generally expanded configuration for collecting debris in a body lumen. To this end, in at least some embodiments, support member 18 may be comprised of a “self-expanding” shape-memory material such as nickel-titanium alloy, which is capable of biasing filter 16 toward being in the second expanded configuration. Additionally, support member 18 may include a radiopaque material or include, for example, a radiopaque wire disposed about a portion thereof. Some further details regarding these and other suitable materials are provided below.

One or more struts 20 may extend between support member 18 and wire 14. Strut 20 may be coupled to wire 14 by a coupling 21. Coupling 21 may be one or more windings of strut 20 about wire 14 or may be a fitting disposed over an end of strut 20 to attach it to wire 14. The exact arrangement of struts 20 can vary considerably. One of ordinary skill in the art would be familiar with the various arrangements of struts 20 that are appropriate for a given intervention.

With filter 16 properly positioned in blood vessel 12, another medical device may be advanced over wire 14 in order to treat and/or diagnose a lesion 28. For example, a catheter 26 (such as the balloon catheter depicted in FIG. 1) may be advanced over wire 14 in order to expand lesion 28. Of course numerous other devices could just as easily be passed over wire 14 including any device designed to pass through an opening or body lumen. For example, the device may comprise any type of catheter (e.g., therapeutic, diagnostic, or guide catheter), a stent delivery catheter, an endoscopic device, a laproscopic device, variations and refinements thereof, and the like, or any other suitable device. Alternatively, another device may be advanced over or through its own guiding structure to a suitable location adjacent filter 16 in a manner that allows device 10 to perform its intended filtering function.

Filtering device 10 is generally designed to filter embolic debris that might be generated during the course of this medical intervention. For example, device 10 can be used to capture embolic debris that might be generated during the use of catheter 26 such as when a balloon 30 (coupled to catheter 26) is inflated. It should be noted, however, that device 10 may find utility in concert with essentially any procedure that has the potential to loosen and release embolic debris in to the blood stream or with the devices associated with such procedures.

Maintaining the position of a filtering device within a blood vessel during an intervention may be desirable. For example, if the filter migrates within the vessel during an intervention, the filter could come into contact with another device (e.g., a catheter disposed on wire 14) and potentially interfere with the goals of the intervention. In addition, advancing other devices over the filter wire may cause small shifts in the position of the filtering device itself that takes the filtering device out of its optimal position. In at least some embodiments, the present invention addresses this potential complication by providing vessel stabilization structures that are incorporated into the design of filtering device 10 and that improve the ability of filtering device 10 to hold and/or maintain its position during an intervention.

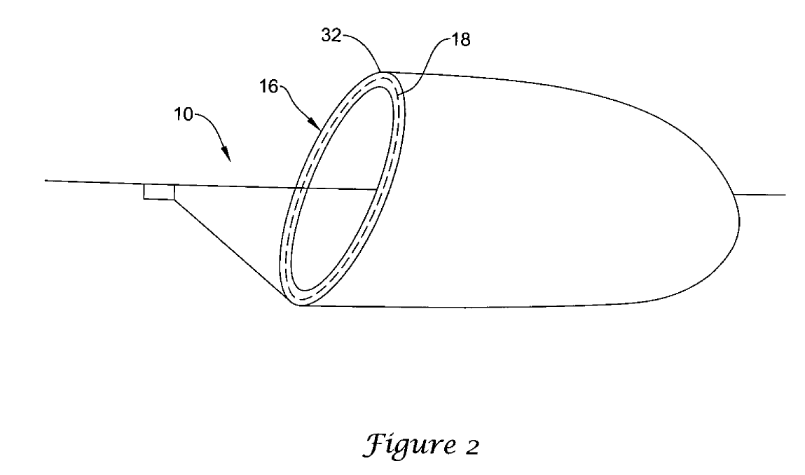

Turning now to FIG. 2, here it can be seen that at least some embodiments of filtering device 10 include a traction member 32 coupled to filter 16, for example, at or adjacent support member 18. Traction member 32 may vary considerable. For example, traction member 32 may take the form of a thickening or thickened portion of support member 18. This creates an increased amount of surface area for contact between support member 18 and the blood vessel wall and improves the bonding between the two. In other embodiments, traction member 32 may include a coating or surface modification that improves the ability of filtering device to hold its position. For example, traction member 32 may include a position-stabilizing coating that is disposed on support member 18. The coating may include a substance that helps stabilize the position of filtering device 10 such as an adhesive, a hydrophobic polymer, an abrasive coating, and the like, or any other suitable coating. This embodiment is represented in FIG. 2 by coating 32 disposed on support member 18, which is shown in phantom. Some further details regarding these and other embodiments are presented below.

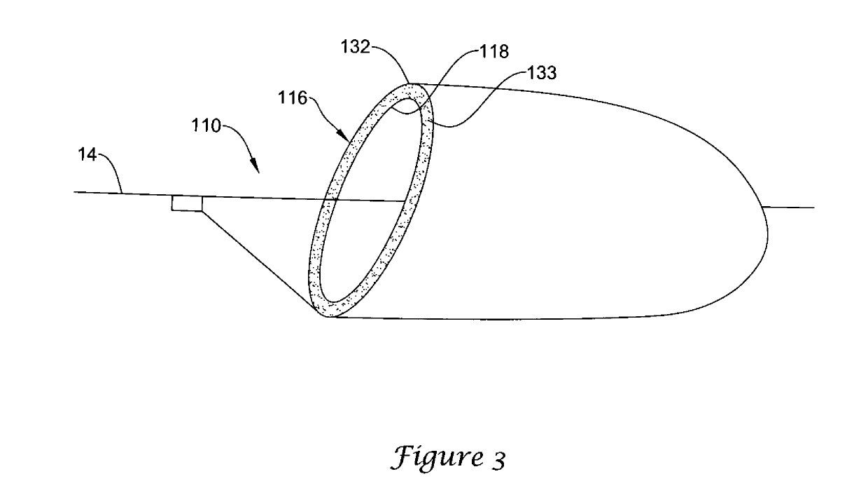

Another example filtering device 110 is illustrated in FIG. 3. Device 110 is similar in form and function to device 10 except that traction member 132, disposed on support member 118 of filter 116 includes a micro-abrasive coating. The micro-abrasive coating generally includes an appropriately roughened or textured outer surface that can “grip” or otherwise engage the wall of a body lumen and hold the longitudinal position of device 110 in the body lumen.

The micro-abrasive traction member 132 may be formed in a number of different ways. For example, the micro-abrasive may be suspended in a coating solution such that application of the coating solution to filter 116 (e.g., at support member 118) places the micro-abrasive onto filter 116. Alternatively, the micro-abrasive may be in an aerosol or sprayable formulation that can be easily spray coated onto filter 116. Of course a number of additional methodologies are contemplated for disposing traction member 132 onto filter 116.

The micro-abrasive substance itself in traction member 132 can also vary. The micro-abrasive substance is understood to be the particles or grains in traction member 132 that give it the micro-abrasive feel. The particles or grains are represented in FIG. 3 as speckles dispersed on traction member 132, for example at reference number 133. These particles 133 are akin to the grains in sandpaper that give sandpaper its particular “feel”. Alternatively, the micro-abrasive grains 133 may include one or more barbs, spikes, teeth, or the like that function by engaging the wall of the body lumen so as to hold device 110 in place.

In some embodiments, the micro-abrasive grains 133 in traction member 132 may be a biologically-compatible polymer, ceramic, silica-based compound, combinations thereof, and the like, or any other suitable material. In other embodiments, the micro-abrasive particles 133 may comprise a biologically active compound such as a pharmaceutical. These later embodiments may be desirable because they allow filtering device 110 to also have drug-delivering capabilities by designing the traction member 132 in such a way that the micro-abrasive “drug” 133 can slowly elute or otherwise come off of filter 116 in the appropriate manner.

It should be noted that although traction member 132 is depicted in FIG. 3 as being a micro-abrasive coating disposed on filter 116, the application of a coating is not the only way that a micro-abrasive can be formed on filter 116. For example, a number of additional embodiments are contemplated where the micro-abrasive is formed by scoring or otherwise modifying the surface texture of filter 116 or support member 1I 8 so as to define a micro-abrasive surface on support member 118. In some of these embodiments, filter 116 (e.g., at support member 118) may be slightly thickened so that the modified surface texture can be formed without compromising the integrity of support member 118. In other embodiments, such thickening is not necessary.

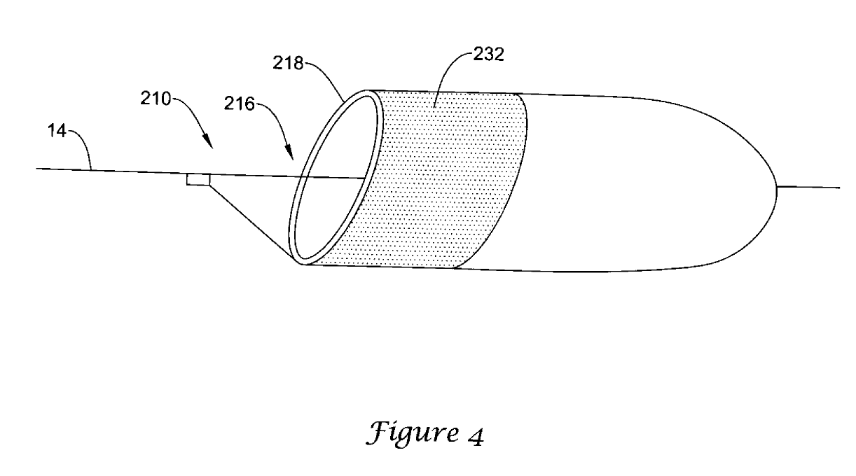

Another example filtering device 210 is shown in FIG. 4. Device 210 is similar to any of the other devices disclosed herein expect that traction member 232 comprises a longitudinal extension of filter 216 that extends from support member 218. The design of traction member 232 creates a greater longitudinal surface of contact between the body lumen (e.g., the blood vessel wall) and filter 216. This increase in surface area contact, alone, improves the stability of filtering device 210 in the vessel.

In some embodiments, traction member 232 further includes a coating or substance that improves the longitudinal position-stabilizing features of device 210. The coating may include a “sticky”, adhesive, or adhesive-like substance that helps hold traction member 232 in place when deployed. Alternatively, traction member 232 may include a hydrophobic polymer or a micro-abrasive coating such as the micro-abrasive described above.

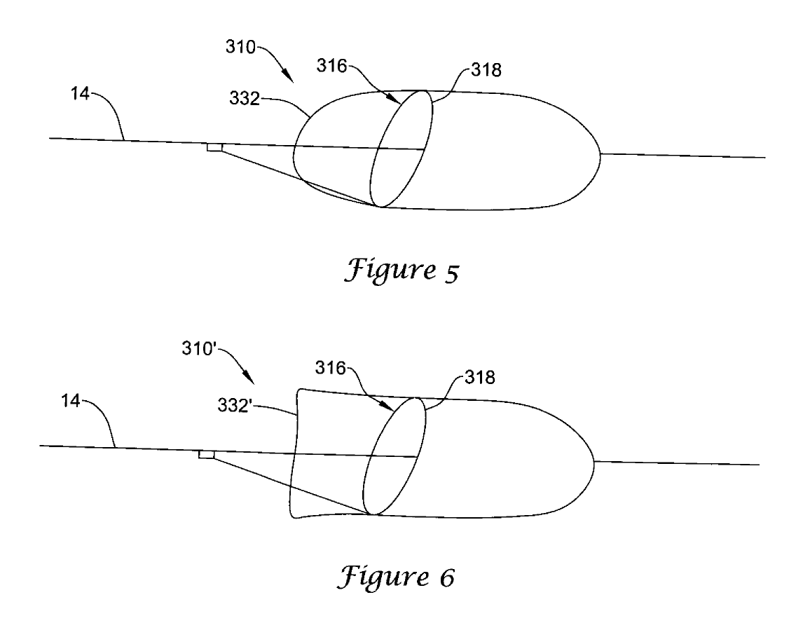

Another example filtering device 310 is shown in FIG. 5. Device 310 is similar to any of the other devices described herein except that traction member 332 comprises a buckling extension member that is coupled to filter 316, for example, at support member 318. Buckling extension member 332 generally is attached to support member 318 and extends proximally therefrom. In some embodiments, buckling extension member 332 may have a generally rounded shape (as depicted in FIG. 5) whereas other embodiments may have a flared or fanned shape. The later embodiment is illustrated in FIG. 6 as flared buckling extension member 332′ that make up part of device 310′.

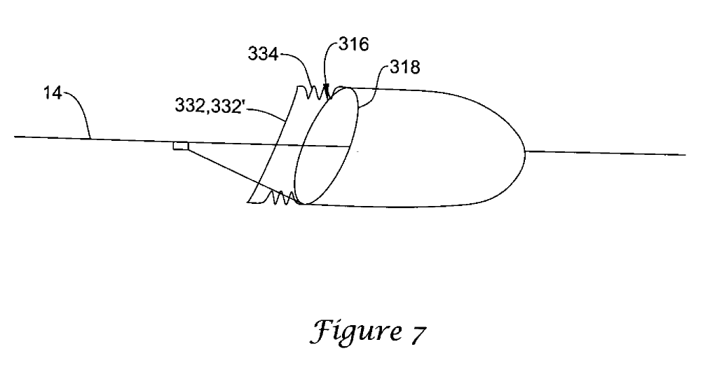

Regardless of the shape, buckling extension member 332/332′ is generally configured to shift between a first non-buckled configuration (e.g., the configurations shown in FIGS. 5-6) and a buckled configuration, which is shown in FIG. 7. The buckled configuration of buckling extension member 332/332′ is generally longitudinally shorter than the non-buckled configuration and includes one or more outward-projecting buckles or ridges 334 that extend radially beyond the diameter of support member 318. Thus, when deployed in a vessel, ridges 334 can project into the wall of a body lumen (e.g., the wall of a blood vessel) so as to “grip” the wall and hold the position of device 310.

The mechanism for activating the shift between the non-buckled and buckled configuration of buckling extension member 332 can vary. In some embodiments, buckling extension member 332 is attached to filter 316 so that if a force is applied to support member 318 (e.g., by the application of force in the proximal direction to wire 14), the force transfers to buckling extension member 332, thereby slightly deforming buckling extension member 332 and causing it to shift between the non-buckled configuration and the buckled configuration. For example, with filtering device 310 deployed in a blood vessel and with buckling extension member 332 in a non-buckled configuration, if the clinician proximally retracts wire 14, the proximal force will transfer from wire 14 to filter 316 to buckling extension member 332, causing buckling extension member 332 to buckle. This stabilizes the position of filtering device 310 in the vessel and helps prevent device 310 from unintentionally drifting proximally to a position where it may interfere with the overall intervention. “Unbuckling”, under this scenario, may be achieved by distally advancing wire 14 or by simply releasing the proximal pressure on wire 14.

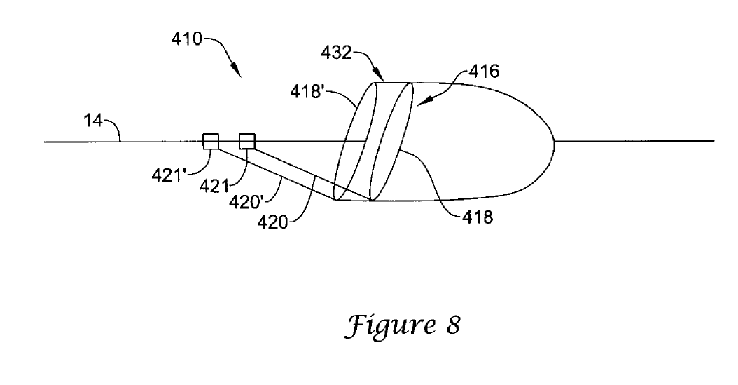

FIG. 8 depicts another example filtering device 410, similar to others disclosed herein, that includes filter 416 coupled to wire 14. Filter 416 includes support member 418, similar to any of the other support members disclosed herein, and a second support member 418′. A first strut 420 extends between support member 418 and wire 14 and is coupled to wire 14 by coupling 421. A second strut 420′ extends between second support member 418′ and wire 14 and is coupled to wire 14 by coupling 421′.

Between support members 418/418′ is buckling extension member 432. Buckling extension member 432 may take any of a number of different forms. For example, buckling extension member 432 may comprise one or more wires extending between support members 418/418′. Alternatively, buckling extension member 432 may be generally cylindrical in shape.

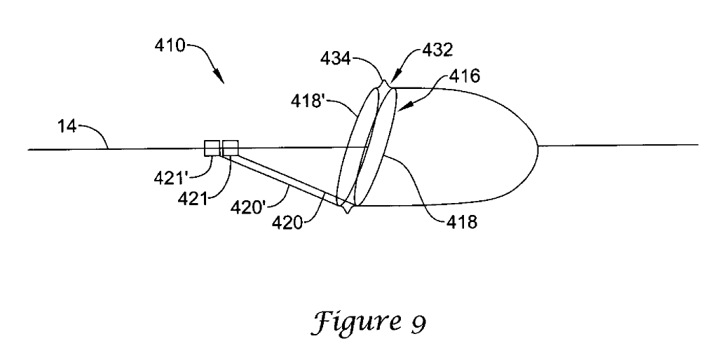

Regardless of the shape or configuration, buckling extension member 432 is generally configured to shift between a first non-buckled configuration (e.g., the configuration shown in FIG. 8) and a buckled configuration, which is shown in FIG. 9, much like buckling extension member 332 (and or member 332′). The buckled configuration of buckling extension member 432 is generally longitudinally shorter than the non-buckled configuration and includes one or more outward-projecting buckles or ridges 434 that extend radially beyond the diameter of support members 418/418′. Thus, when deployed in a vessel, ridges 434 can project into the wall of a body lumen (e.g., the wall of a blood vessel) so as to “grip” the wall and hold the position of device 410.

The mechanism for activating the shift between the non-buckled and buckled configuration of buckling extension member 432 can vary. In some embodiments, one or both of couplings 421/421′ (e.g., coupling 421′) may be slidable along wire 14. This allows a user to shift buckling extension member 432 between the buckled and non-buckled configuration. For example, with filtering device 410 deployed in a blood vessel and with buckling extension member 432 in a non-buckled configuration, if the clinician proximally retracts wire 14, coupling 421′ may slide distally along wire 14 to a position nearer to coupling 421, causing and/or allowing buckling extension member 432 to buckle. This stabilizes the position of filtering device 410 in the vessel and helps prevent device 410 from unintentionally drifting proximally to a position where it may interfere with the overall intervention. “Unbuckling”, under this scenario, may be achieved by distally advancing wire 14 to allow coupling 421′ to shift proximally away from coupling 421.

The overall design of filtering devices 10/110/210/3 10/310′/410 disclosed herein includes the use of a number of different materials appropriate for the various components thereof. These materials may include metals, metal alloys, polymers, metal-polymer composite, and the like, or any other suitable material. Some examples of suitable metals and metal alloys include stainless steel, such as 304V, 304L, and 316LV stainless steel; mild steel; nickel-titanium alloy such as linear-elastic or super-elastic nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like; other Co—Cr alloys; platinum enriched stainless steel; or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In addition, filtering devices 10/110/210/310/310′/410 or portions thereof, may also be doped with or otherwise include a radiopaque material as stated above in relation to support member 18. Radiopaque materials are understood to be materials capable of producing a relatively bright image on a fluoroscopy screen or another imaging technique during a medical procedure. This relatively bright image aids the user of filtering devices 10/110/210/310/310′ in determining their location. Some examples of radiopaque materials can include, but are not limited to, gold, platinum, molybdenum, palladium, tantalum, tungsten or tungsten alloy, plastic material loaded with a radiopaque filler, and the like.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An embolic protection filtering device, comprising:
an elongate shaft;
a filter coupled to the shaft, the filter including a support member disposed about the shaft and a strut region extending between the support member and the shaft;
a filter membrane coupled to the support member, the filter membrane having a plurality of openings defined therein; and
a traction member coupled to the support member.

2. The filtering device of claim 1, wherein the traction member includes a coating disposed on the support member.

3. The filtering device of claim 2, wherein the coating includes a micro-abrasive.

4. The filtering device of claim 2, wherein the coating includes a hydrophobic polymer.

5. The filtering device of claim 2, wherein the coating includes an adhesive.

6. The filtering device of claim 1, wherein the traction member includes a micro-abrasive formed on the filter.

7. The filtering device of claim 6, wherein the micro-abrasive is formed on the support member.

8. The filtering device of claim 6, wherein the micro-abrasive includes one or more barbs.

9. The filtering device of claim 6, wherein the micro-abrasive includes one or more spikes.

10. The filtering device of claim 6, wherein the micro-abrasive includes one or more teeth.

11. The filtering device of claim 6, wherein the micro-abrasive includes a pharmaceutical substance.

12. The filtering device of claim 1, wherein the traction member includes a longitudinal extension of the support member.

13. The filtering device of claim 1, wherein the traction member includes a buckling extension member that is attached to the support member and extends proximally therefrom.

14. The filtering device of claim 13, wherein the buckling extension member has a substantially rounded shape.

15. The filtering device of claim 13, wherein the buckling extension member has a substantially flared shape.

16. The filtering device of claim 13, wherein the buckling extension member is configured to shift between a first non-buckled configuration and a second buckled configuration.

17. The filtering device of claim 16, wherein the buckling extension member is adapted to shift between the non-buckled configuration and the buckled configuration when force is applied to the filter wire.

18. The filtering device of claim 16, wherein the buckling extension member includes one or more ridges when buckling extension member is in the buckled configuration.

19. The filtering device of claim 16, wherein the buckling extension member is disposed between the support member and a second support member.

20. An embolic protection filtering device, comprising:
an elongate filter wire;
a filter coupled to the filter wire, the filter including a support member, a strut extending between the support member and the filter wire, and a filter membrane coupled to the support member and extending therefrom; and
a position-stabilizing traction member coupled to the support member, the traction member being configured to secure the position of the filtering device when the filtering device is deployed in a target region.

21. The filtering device of claim 20, wherein the traction member includes a coating disposed on the support member.

22. The filtering device of claim 20, wherein the traction member includes a micro-abrasive formed on the support member.

23. The filtering device of claim 22, wherein the micro-abrasive includes a pharmaceutical substance.

24. The filtering device of claim 20, wherein the traction member includes a longitudinal extension of the support member.

25. The filtering device of claim 20, wherein the traction member includes a buckling extension member that is attached to the support member and extends proximally therefrom.

26. The filtering device of claim 25, wherein the buckling extension member has a substantially rounded shape.

27. The filtering device of claim 25, wherein the buckling extension member has a substantially flared shape.

28. The filtering device of claim 25, wherein the buckling extension member is configured to shift between a first non-buckled configuration and a second buckled configuration.

29. The filtering device of claim 28, wherein the buckling extension member is adapted to shift between the non-buckled configuration and the buckled configuration when force is applied to the filter wire.

30. The filtering device of claim 28, wherein the buckling extension member includes one or more ridges when buckling extension member is in the buckled configuration.

31. An embolic protection filtering device, comprising:
an elongate filter wire;
a filter coupled to the filter wire, the filter including a support member, a strut extending between the support member and the filter wire, and a filter membrane coupled to the support member and extending therefrom; and
means for longitudinally stabilizing the position of the filtering device, wherein the means for longitudinally stabilizing the position of the filtering device is coupled to the support member.
